(19) United States
(12) Patent Application Publication
(10) Pub. No.: US 2006/0094941 A1
(43) Pub. Date: May 4, 2006

(54) Optical measurement apparatus and blood sugar level measuring apparatus using the same

(76) Inventors: Ok-Kyung Cho, Schwerte (DE); Yoon-Ok Kim, Schwerte (DE); Nobuhiko Sato, Iruma (JP); Hiroshi Mitsumaki, Tokyo (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 10/975,492

(22) Filed: Oct. 29, 2004

(51) Int. Cl. A61B 5/00

(52) U.S. Cl. 600/316; 600/365; 600/549

(57) ABSTRACT

An apparatus for non-invasively measuring blood sugar levels based on temperature measurements. A blood sugar level non-invasively measured by a temperature measuring method is corrected by blood oxygen saturation and blood flow volume. Optical sensors detect scattered light, reflected light, and light exiting from a body surface after penetrating the skin, so that measurement data can be stabilized by taking into consideration the influence of the thickness of the skin on blood oxygen saturation.

### CO-PENDING APPLICATION

U.S. patent application Ser. No. 10/620,689 is a co-pending application of this application. The disclosures of the co-pending application are incorporated herein by cross-reference.

### BACKGROUND OF THE INVENTION

1. Field of the Invention

The present invention relates to non-invasive measurement of blood sugar levels for measuring glucose concentration in a living body without blood sampling, and an optical measurement apparatus suitable therefor.

2. Description of Related Art

Hilson et al. report facial and sublingual temperature changes in diabetics following intravenous glucose injection (Diabete & Metabolisme, “Facial and sublingual temperature changes following intravenous glucose injection in diabetics” by R. M. Hilson and T. D. R. Hockaday, 1982, 8, 15-19). Scott et al. discuss the issue of diabetics and thermoregulation (Can. J. Physiol. Pharmacol., “Diabetes mellitus and thermoregulation”, by A. R. Scott, T. Bennett, I. A. MacDonald, 1987, 65, 1365-1376). Based on the knowledge gained from such researches, Cho et al. suggest a method and apparatus for determining blood glucose concentration by temperature measurement without requiring the collection of a blood sample (U.S. Pat. Nos. 5,924,996, and 5,795,305).

Various other attempts have been made to determine glucose concentration without blood sampling. For example, a method has been suggested (JP Patent Publication (Kokai) No. 2000-258343 A) whereby a measurement site is irradiated with near-infrared light of three wavelengths, and the intensity of transmitted light as well as the temperature of the living body is detected. A representative value of the second-order differentiated value of absorbance is then calculated, and the representative value is corrected in accordance with the difference between the living body temperature and a predetermined reference temperature. The blood sugar concentration corresponding to the thus corrected representative value is then determined. An apparatus is also provided (JP Patent Publication (Kokai) No. 10-33512 A (1998)) whereby a measurement site is heated or cooled while monitoring the living body temperature. The degree of attenuation of light based on light irradiation is measured at the moment of temperature change so that the glucose concentration responsible for the temperature-dependency of the degree of light attenuation can be measured. Further, an apparatus is reported (JP Patent Publication (Kokai) No. 10-108857 A (1998)) whereby an output ratio between reference light and transmitted light following the irradiation of the sample is taken, and then a glucose concentration is calculated in accordance with a linear expression of the logarithm of the output ratio and the living body temperature. Another apparatus for measuring glucose concentration is provided (U.S. Pat. No. 5,601,079) whereby the result of irradiation using two light sources is detected by three infrared light detectors and also temperature is detected.

### SUMMARY OF THE INVENTION

Glucose (blood sugar) in blood is used for glucose oxidation reaction in cells to produce necessary energy for the maintenance of living bodies. In the basal metabolism state, in particular, most of the produced energy is converted into heat energy for the maintenance of body temperature. Thus, it can be expected that there is some relationship between blood glucose concentration and body temperature. However, as is evident from the way sicknesses cause fever, the body temperature also fluctuates due to factors other than blood glucose concentration. While methods have been proposed to determine blood glucose concentration by temperature measurement without blood sampling, they could hardly be considered sufficiently accurate.

Further, a method has also been proposed that detects the result of irradiation of light from two light sources using three infrared light detectors, and that also detects temperature for determining glucose concentration. The method, which only detects two kinds of optical intensity, is unable to provide sufficient accuracy.

It is an object of the invention to provide a method and apparatus for determining blood glucose concentration with high accuracy based on temperature data and optical data of a test subject without blood sampling.

Blood sugar is delivered to the cells throughout the human body via blood vessel systems, particularly the capillary blood vessels. In the human body, complex metabolic pathways exist. Glucose oxidation is a reaction in which, fundamentally, blood sugar reacts with oxygen to produce water, carbon dioxide, and energy. Oxygen herein refers to the oxygen delivered to the cells via blood. The volume of oxygen supply is determined by the blood hemoglobin concentration, the hemoglobin oxygen saturation, and the volume of blood flow. On the other hand, the heat produced in the body by glucose oxidation is dissipated from the body by convection, heat radiation, conduction, and so on. On the assumption that the body temperature is determined by the balance between the amount of energy produced in the body by glucose burning, namely heat production, and heat dissipation such as mentioned above, the inventors set up the following model:

(1) The amount of heat production and the amount of heat dissipation are considered equal.

(2) The amount of heat production is a function of the blood glucose concentration and the volume of oxygen supply.

(3) The volume of oxygen supply is determined by the blood hemoglobin concentration, the blood hemoglobin oxygen saturation, and the volume of blood flow in the capillary blood vessels.

(4) The amount of heat dissipation is mainly determined by heat convection and heat radiation.

According to this model, we achieved the present invention after realizing that blood sugar levels can be accurately determined on the basis of the results of measuring the temperature of the body surface and parameters relating to the blood oxygen concentration and the blood flow volume. The parameters can be measured, e.g., from a part of the human body, such as the fingertip. The parameters relating to convection and radiation can be determined by measuring the temperature on the fingertip. The parameters relating to the blood hemoglobin concentration and the blood hemoglobin oxygen saturation can be determined by spectroscopically measuring blood hemoglobin and then finding the ratio between hemoglobin bound with oxygen and hemoglobin not bound with oxygen. The parameter relating to the volume of blood flow can be determined by measuring the amount of heat transfer from the skin.

The invention provides an optical measurement apparatus comprising: a first light source for producing light of a first wavelength; a first optical fiber for irradiating a light incident point on the surface of a subject with the light from said first light source; a second light source for producing light of a second wavelength; a second optical fiber for irradiating said light incident point on the surface of the subject with the light from said second light source in a direction different from that of the light of said first wavelength; a first photodetector on which reflected light of the light of said first wavelength reflected by said light incident point and scattered light of the light of said second wavelength are incident without via fiber; a second photodetector on which reflected light of the light of said second wavelength reflected at said light incident point and scattered light of the light of said first wavelength are incident without via fiber; a third photodetector; and a third optical fiber having an incident end thereof disposed at such a position as to be in contact with the surface of said subject, said third optical fiber being adapted to receive, on an incident end thereof, light exiting from an area spaced apart from said light incident point on the surface of said subject and then transmit the light to said third detector.

Preferably, the plane of incidence of the light of the first wavelength and the plane of incidence of the light of the second wavelength are substantially perpendicular to each other with respect to the light incident point on the subject surface. The plane of incidence herein refers to a plane that includes the incident ray and a normal at the incident point on the subject surface. Further, in the present specification, the ray that enters the incident plane after having been irradiated onto the incident point on the subject surface will be referred to as reflected light. The light that leaves in directions other than that of the incident plane from near the incident point will be referred to as scattered light. The scattered light that leaves out of a position on the subject surface that is spaced apart from the incident point will be referred to as traveled photon.

Preferably, the outgoing light from each light source is irradiated onto the light incident point on the subject surface via an optical fiber. The reflected light and scattered light from the examined subject are directly incident on the photodetector, and the traveled photon is incident on the photodetector via an optical fiber. An exiting end of the light-irradiating optical fiber and an incident end of the optical fiber for detecting reflected or scattered are preferably disposed near the plane of a cone whose apex corresponds to the light incident point on the subject surface. The first wavelength may be a wavelength at which the molar absorption coefficient of oxyhemoglobin is equal to that of reduced hemoglobin, and the second wavelength may be a wavelength for detecting the difference in absorbance between the oxyhemoglobin and reduced hemoglobin.

The invention further provides a blood sugar level measuring apparatus comprising: (1) a heat-amount measuring portion for measuring a plurality of temperatures deriving from a body surface and acquiring information that is used for calculating a convective heat transfer amount and radiation heat transfer amount that are related to the dissipation of heat from the body surface; (2) a blood flow volume measuring portion for acquiring information about the volume of blood flow; (3) an optical measurement portion including a light source for producing light of at least two different wavelengths, an optical system for irradiating the body surface with the light emitted by said light source, and at least three different photodetectors for detecting the light that has been irradiated onto the body surface, said optical measurement portion providing hemoglobin concentration and hemoglobin oxygen saturation in blood; (4) a memory portion in which relationships between parameters respectively corresponding to said plurality of temperatures, blood flow volume, hemoglobin concentration and hemoglobin oxygen saturation in blood, and blood sugar levels are stored; (5) a calculation portion for converting a plurality of measurement values inputted from said heat amount measuring portion, said blood flow volume measuring portion, and said optical measurement portion respectively into said parameters, and then calculating a blood sugar value by applying said parameters to said relationships stored in said memory portion; and (6) a display portion for displaying the blood sugar level calculated by said calculation portion, wherein said optical measurement portion includes a first light source for producing light of a first wavelength, a second light source for producing light of a second wavelength, a first optical fiber, a second optical fiber, a third optical fiber, a first photodetector, a second photodetector, and a third photodetector, a light incident point on the surface of a subject is irradiated with light emitted by said first light source via said first optical fiber, said light incident point on the surface of said subject is irradiated with light emitted by said second light source via said second optical fiber in a direction different from that of the light of said first wavelength, reflected light of the light of said first wavelength reflected at said light incident point and scattered light of the light of said second wavelength are incident on said first photodetector without via fiber, reflected light of the light of said second wavelength reflected at said light incident point and scattered light of the light of said first wavelength are incident on said second photodetector without via fiber, said third optical fiber has an incident end thereof disposed at such a position as to be in contact with the surface of said subject in an area spaced apart from said light incident point on the subject surface, and said third photodetector is adapted to receive light exiting from an area spaced apart from said light incident point on the surface of the subject via said third optical fiber.

### BRIEF DESCRIPTION OF THE DRAWINGS

FIG. 1 shows a model of the transmission of light in the case of irradiating the skin surface with continuous light.

FIG. 2 shows a model of heat transfer from the body surface to a block.

FIG. 3 shows a temporal change in measurement values of temperatures T₁ and T₂.

FIG. 4 shows an example of a measurement of a temporal change in temperature T₃.

FIG. 5 shows the relationships between measurement values provided by various sensors and the parameters derived therefrom.

FIG. 6 shows an upper plan view of a non-invasive blood sugar level measuring apparatus according to the present invention.

FIG. 7 shows the operating procedure for the apparatus.

FIGS. 8A to 8E show a measuring portion in detail.

FIG. 9 shows a block diagram of an example of a circuit for causing light-emitting diodes to emit light in a time-divided manner.

FIGS. 10A to 10G show an optical sensor portion and the measuring portion in detail.

FIGS. 11A to 11B show in detail the measuring portion for a plurality of wavelengths.

FIGS. 12A to 12D show in detail the measuring portion for a plurality of wavelengths.

FIG. 13 shows the connection and blocking of light between a light-emitting diode and an optical fiber.

FIG. 14 shows a conceptual chart illustrating the flow of data processing in the apparatus.

FIG. 15 shows a chart plotting the glucose concentration values calculated according to the present invention and the glucose concentration values measured by the enzymatic electrode method.

### DETAILED DESCRIPTION OF THE PREFERRED EMBODIMENTS

The invention will now be described by way of preferred embodiments thereof with reference made to the drawings.

Initially, the above-mentioned model will be described in more specific terms. Regarding the amount of heat dissipation, convective heat transfer, which is one of the main causes of heat dissipation, is related to temperature difference between the ambient (room) temperature and the body-surface temperature. The amount of heat dissipation due to radiation, which is another main cause of dissipation, is proportional to the fourth power of the body-surface temperature according to the Stefan-Boltzmann law. Thus, it can be seen that the amount of heat dissipation from the human body is related to the room temperature and the body-surface temperature. On the other hand, the amount of oxygen supply, which is a major factor related to the amount of heat production, is expressed as the product of hemoglobin concentration, hemoglobin oxygen saturation, and blood flow volume.

The hemoglobin concentration can be measured from the absorbance at the wavelength (equal-absorbance wavelength) at which the molar absorbance coefficient of the oxyhemoglobin is equal to that of the reduced (deoxy-) hemoglobin. The hemoglobin oxygen saturation can be measured by measuring the absorbance at the equal-absorbance wavelength and the absorbance at at least one different wavelength at which the ratio between the molar absorbance coefficient of the oxyhemoglobin and that of the reduced (deoxy-) hemoglobin is known, and then solving simultaneous equations. Namely, the hemoglobin concentration and hemoglobin oxygen saturation can be obtained by conducting the measurement of absorbance at at least two wavelengths. However, in order to accurately determine the hemoglobin concentration and hemoglobin oxidation saturation from absorbance, the influence of interfering components must be corrected. The interfering components affecting the absorbance include the thickness of the skin (epidermis), for example. These interfering components can be measured in various manners, of which one example will be described below.

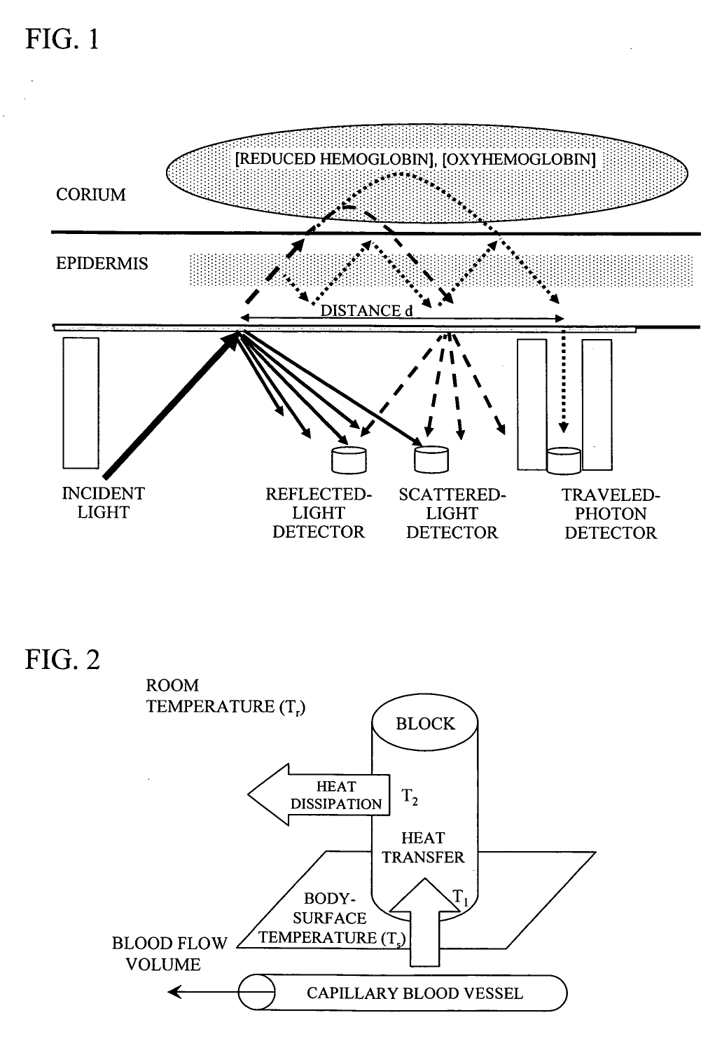

The thickness of the skin can be measured by measuring the intensity of only that light that has traveled in the skin by a distance d from where light was shone on the skin. FIG. 1 shows the behavior of light in the case where the skin surface was irradiated with continuous light. As the light of a certain wavelength and intensity is shone, the light is reflected and scattered by the skin surface. Part of the light penetrates the skin and experiences scattering and diffusion in a repeated manner. In such a behavior of light, the depth of penetration of the light that has traveled by distance d is substantially constant depending on the wavelength. The skin does not contain blood, so it has a low fluidity, resulting in a low absorbance. On the other hand, the corium contains blood and therefore has a high fluidity, resulting in a high absorbance. Thus, when the skin is thin, the light can penetrate deeper into the corium, resulting in a larger absorbance. When the skin is thick, the distance traveled by the light becomes shorter, so that the absorbance becomes smaller. By taking the ratio between the detected intensity of only that light that has traveled distance d and the detected intensity of the light that has traveled in a standard substance with a known thickness in the same manner, the thickness of the skin can be estimated.

The measurements are carried out using at least three detectors, namely a reflected-light detector for detecting mainly reflected light, a scattered-light detector for detecting mainly scattered light, and a traveled-photon detector for detecting traveled photon.

The reflected-light detector can detect part of the scattered light produced by the light traveling inside the body and then exiting from the body surface, as well as mainly the reflected light reflected by the body surface. The scattered-light detector can detect part of the scattered light scattered on the body surface, as well as mainly the scattered light produced by the light passing inside the body and then exiting through the body surface. The path of the traveled photon up to the traveled-photon detector is optically blocked in order to prevent the detection of light other than the traveled photon, namely the light deriving from reflected light and scattered light. The traveled-photon detector is thus adapted to detect only traveled photon, so that the skin thickness can be estimated. During detection, a total of at least three detectors, namely at least one each of the reflected-light detector, scattered-light detector, and traveled-photon detector, are used. Preferably, additional detectors with similar functions and high detection sensitivities adapted for particular kinds of wavelength may be used. Further, a transmitted-light detector may be added for detecting light that has passed through the detection area, as necessary.

The wavelength values described herein are most appropriate values for obtaining absorbance for various intended purposes, such as for obtaining the absorbance at the equal molar absorbance coefficient wavelengths, or for obtaining the peak of absorbance. Thus, in addition to the wavelengths described herein, other wavelengths in the vicinities thereof may be used and still similar measurements can be performed.

The rest is the blood flow volume, which can be measured by various methods. One example will be described below.

FIG. 2 shows a model for the description of the transfer of heat from the body surface to a solid block having a certain heat capacity when the block is brought into contact with the body surface for a certain time and then separated. The block is made of resin such as plastic or vinyl chloride. In the illustrated example, attention will be focused on the temporal variation of the temperature T₁ of a portion of the block that is brought into contact with the body surface, and the temporal variation of the temperature T₂ at a point on the block spaced apart from the body surface. The blood flow volume can be estimated by monitoring mainly the temporal variation of the temperature T₂ (at the spatially separated point on the block). The details will follow.

Before the block comes into contact with the body surface, the temperatures T₁ and T₂ at the two points of the block are equal to the room temperature Tᵣ. When a body-surface temperature Tₛ is higher than the room temperature Tᵣ as the block comes into contact with the body surface, the temperature T₁ swiftly rises due to the transfer of heat from the skin, and it approaches the body-surface temperature Tₛ. On the other hand, the temperature T₂ is lowered from the temperature T₁ as the heat conducted through the block is dissipated from the block surface, and it rises more gradually. The temporal variation of the temperatures T₁ and T₂ depends on the amount of heat transferred from the body surface to the block, which in turn depends on the blood flow volume in the capillary blood vessels under the skin. If the capillary blood vessels are regarded as a heat exchanger, the coefficient of transfer of heat from the capillary blood vessels to the surrounding cell tissues is given as a function of the blood flow volume. Thus, by measuring the amount of heat transfer from the body surface to the block by monitoring the temporal variation of the temperatures T₁ and T₂, the amount of heat transferred from the capillary blood vessels to the cell tissues can be estimated. Based on this estimation, the blood flow volume can then be estimated.

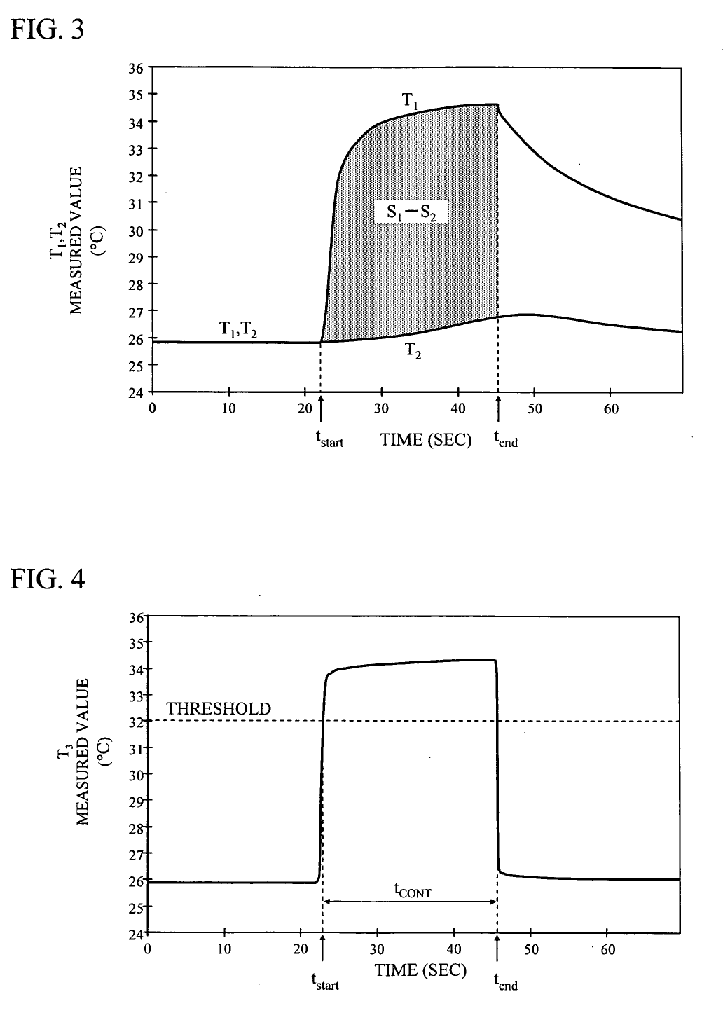

FIG. 3 shows the temporal variation of the measured values of the temperature T₁ at the portion of the block in contact with the body surface and the temperature T₂ at the position on the block spaced apart from the body-surface contact position. As the block comes into contact with the body surface, the T₁ measured value swiftly rises, and it gradually drops as the block is brought out of contact.

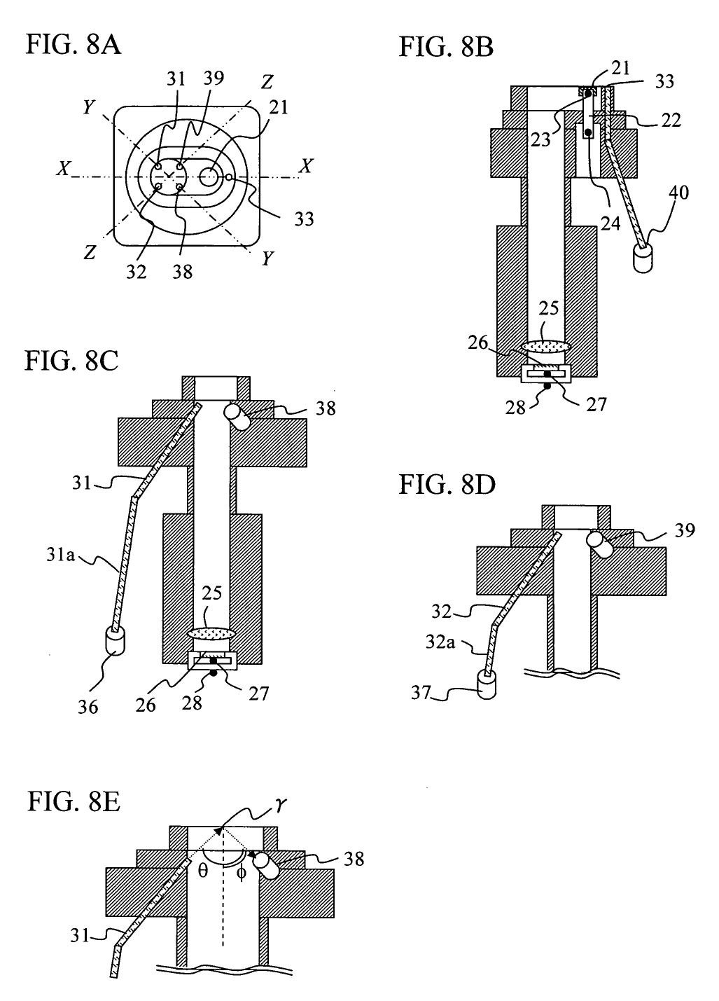

FIG. 4 shows the temporal variation of the value of the temperature T₃ measured by a radiation-temperature detector. As the detector detects the temperature T₃ that is due to radiation from the body surface, it is more sensitive to temperature changes than other sensors. Because radiation heat propagates as an electromagnetic wave, it can transmit temperature changes instantaneously. Thus, by locating the radiation-temperature detector near where the block contacts the body surface so as to detect radiated heat from the body surface, as shown in FIG. 8 (which will be described later), the time of start of contact tₛₜₐᵣₜ and the time of end of contact tend between the block and the body surface can be detected from changes in the temperature T₃. For example, a temperature threshold value is set as shown in FIG. 4. The contact start time tₛₜₐᵣₜ is when the temperature threshold value is exceeded. The contact end time tend is when the temperature T₃ drops below the threshold. The temperature threshold is set at 32° C., for example.

Then, the T₁ measured value between tₛₜₐᵣₜ and tend is approximated by an S curve, such as a logistic curve. A logistic curve is expressed by the following equation:

$$T = \frac{b}{1 + c \times exp ( - a \times t )} + d$$

where T is temperature, and t is time.

The measured value can be approximated by determining coefficients a, b, c, and d using the non-linear least-squares method. For the resultant approximate expression, T is integrated between time tₛₜₐᵣₜ and time tend to obtain a value S₁.

Similarly, an integrated value S₂ is calculated from the T₂ measured value. The smaller (S₁−S₂) is, the larger the amount of transfer of heat is from the body surface to the position of T₂. (S₁−S₂) becomes larger with increasing body-surface contact time t_{CONT} (=t_{end}−tₛₜₐᵣₜ). Thus, a₅/(t_{CONT}×(S₁−S₂)) is designated as a parameter X₅ indicating the volume of blood flow, using a₅ as a proportionality coefficient.

Thus, it will be seen that the measured amounts necessary for the determination of blood glucose concentration by the above-described model are the room temperature (ambient temperature), body surface temperature, temperature changes in the block brought into contact with the body surface, the temperature due to radiation from the body surface, the absorbance of reflected light or scattered light at at least two wavelengths, and the intensity of traveled photon.

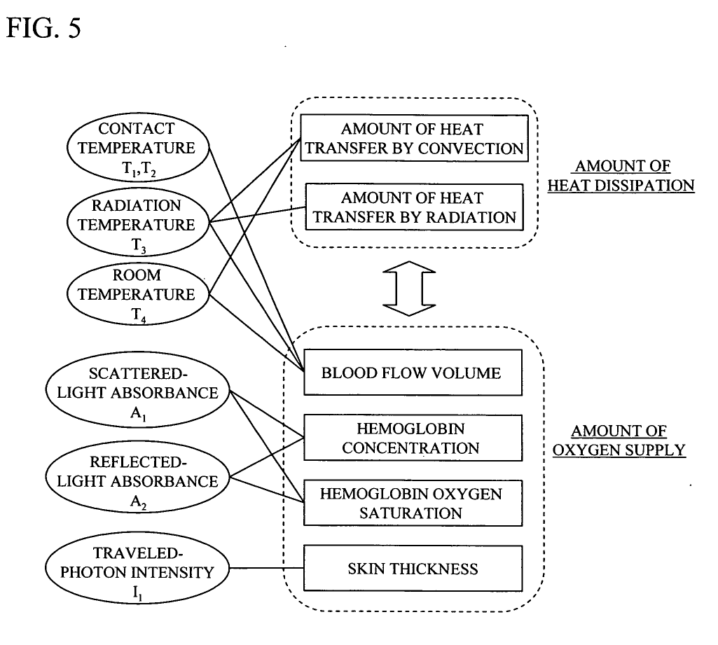

FIG. 5 shows the relationships between the measured values provided by various sensors and the parameters derived therefrom. A block is brought into contact with the body surface, and chronological changes in two kinds of temperatures T₁ and T₂ are measured by two temperature sensors provided at two locations of the block. Separately, radiation temperature T₃ on the body surface and room temperature T₄ are measured. Absorbance A₁ and A₂ of scattered light and reflected light, respectively, are measured at at least two wavelengths related to the absorption of hemoglobin. The intensity I₁ of traveled photon is measured at at least one wavelength. Alternatively, the intensity may be determined by measuring at the aforementioned two wavelengths, and then finding an averaged or mean value from the results thereof. The temperatures T₁, T₂, T₃, and T₄ provide parameters related to the volume of blood flow. The temperature T₃ provides a parameter related to the amount of heat transferred by radiation. The temperatures T₃ and T₄ provide parameters related to the amount of heat transferred by convection. The absorbance A₁ and A₂ and intensity I₁ provide parameters related to the hemoglobin concentration and the hemoglobin oxygen saturation.

Hereafter, an example of an apparatus for non-invasively measuring blood sugar levels according to the principle of the invention will be described.

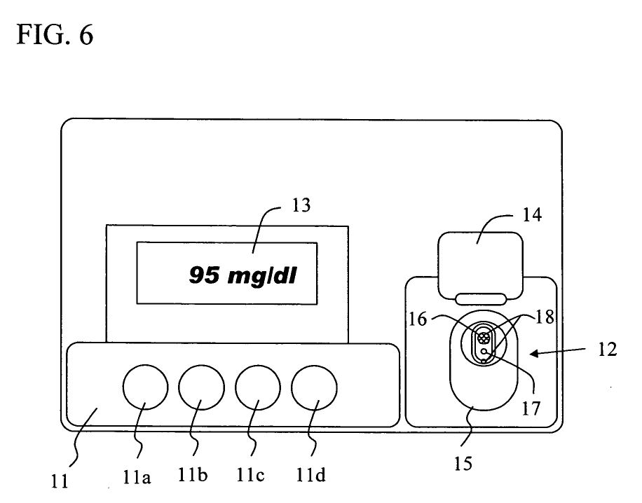

FIG. 6 shows a top plan view of a non-invasive blood sugar level measuring apparatus according to the invention. While in this example the skin on the ball of the fingertip is used as the body surface, other parts of the body surface may be used.

On the top surface of the apparatus are provided an operating portion 1, a measuring portion 12 where the finger to be measured is to be placed, and a display portion 13 for displaying measurement results, the state of the apparatus, measured values, for example. The operating portion 11 includes four push buttons 11a to 11d for operating the apparatus. The measuring portion 12 has a cover 14 which, when opened (as shown), reveals a finger rest portion 15 with an oval periphery. The finger rest portion 15 accommodates an opening end 16 of a radiation-temperature sensor portion, a contact-temperature sensor portion 17, and an optical sensor portion 18.

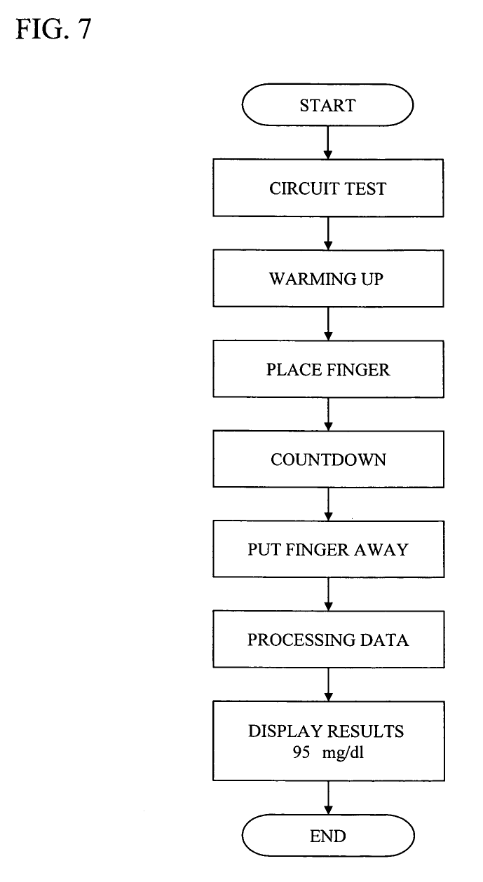

FIG. 7 shows the procedure for operating the apparatus. As a power button on the operating portion is pressed to turn on the apparatus, an indication “Warming up” is displayed on the LCD and the electronic circuits in the apparatus are warmed up. At the same time, a check program is activated to automatically check the electronic circuits. After the warm-up phase is finished, an indication “Place finger” appears on the LCD. As the user places his or her finger on the finger rest portion, a countdown is displayed on the LCD. When the countdown is over, an indication “Put finger away” appears on the LCD. As the user puts his or her finger away, the LCD indicates “Processing data.” Thereafter, the display shows a blood sugar level, which is then stored in an IC card together with the date and time. After the user reads the displayed blood sugar level, he or she pushes a particular button on the operating portion. About one minute later, the apparatus displays a message “Place finger” on the LCD, thus indicating that the apparatus is ready for the next cycle of measurement.

FIGS. 8A to 8E show the measuring portion in detail. FIG. 8A is a top plan view, FIG. 8B is a cross section taken along line X-X of FIG. 8A, FIG. 8C is a cross section taken along line Y-Y of FIG. 8A, and FIG. 8D is a cross section taken along Z-Z of FIG. 8A.

First, the process of measuring temperatures by the non-invasive blood sugar level measuring apparatus according to the invention will be described. In a portion of the measuring portion with which the examined portion (ball of the finger) is to come into contact, a thin plate 21 of a highly heat-conductive material, such as gold, is placed. A bar-shaped heat-conductive member 22, which is made of a material with a heat conductivity lower than that of the plate 21, such as polyvinylchloride, is thermally connected to the plate 21 and extends into the apparatus. The temperature sensors include a thermistor 23 that is an adjacent-temperature detector with respect to the examined portion for measuring the temperature of the plate 21, and a thermistor 24 that is an indirect-temperature detector with respect to the examined portion for measuring the temperature of a portion of the heat-conducting member which is spaced apart from the plate 21 by a certain distance. An infrared lens 25 is disposed inside the apparatus at such a position that the examined portion (ball of the finger) placed on the finger rest portion 15 can be seen through the lens. Below the infrared lens 25 is disposed a pyroelectric detector 27 via an infrared radiation-transmitting window 26. Another thermistor 28 is disposed in close proximity to the pyroelectric detector 27.

Thus, the temperature sensor portion of the measuring portion has four temperature sensors, and they measure four kinds of temperatures as follows:

(1) Temperature on the finger surface (thermistor 23): T₁

(2) Temperature of the heat-conducting member (thermistor 24): T₂

(3) Temperature of radiation from the finger (pyroelectric detector 27): T₃

(4) Room temperature (thermistor 28): T₄

The optical sensor portion 18 is described hereafter. The optical sensor portion 18 measures the hemoglobin concentration and the hemoglobin oxygen saturation necessary for the determination of the oxygen supply volume. In order to measure the hemoglobin concentration and the hemoglobin oxygen saturation, it is necessary to measure the absorbance of scattered light at at least two wavelengths, the absorbance of reflected light at at least one wavelength, and the intensity of traveled photon at at least one wavelength. The accuracy of the absorbance of reflected light can be improved by measuring at a plurality of wavelengths, if possible, and then using a mean value. Thus, in the present embodiment, the absorbance of reflected light is measured at two different wavelengths. The accuracy of the measurement of the intensity of traveled photon can also be improved by measuring at a plurality of wavelengths, if possible, and then using a mean value. FIGS. 8B to 8E show exemplary configurations for carrying out the measurement using two light sources 36 and 37 and three detectors 38 to 40.

The ends of three optical fibers 31 to 33 are located in the optical sensor portion 18. The optical fibers 31 and 32 are for optical irradiation, while the optical fiber 33 is for receiving light. As shown in FIG. 8C, the optical fiber 31 connects to a branch optical fiber 31a that is provided with a light-emitting diode 36 of a single wavelength at the end thereof. Similarly, the optical fiber 32 is connected to a branch optical fiber 32a that is provided at the end thereof with a light-emitting diode 37 of a single wavelength. Photodiodes 38 and 39 are disposed in the optical sensor portion. The other end of the light-receiving optical fiber 33 is provided with a photodiode 40. To each the optical fibers 31 and 32, there may be connected a plurality of branch optical fibers provided at the ends thereof with light-emitting diodes. The light-emitting diode 36 emits light with a wavelength of 810 nm, while the light-emitting diode 37 emits light with a wavelength of 950 nm. The wavelength 810 nm is the equal absorbance wavelength at which the molar absorbance coefficient of the oxyhemoglobin is equal to that of the reduced (deoxy-) hemoglobin. The wavelength 950 nm is the wavelength at which the difference between the molar absorbance coefficient of the oxyhemoglobin and that of the reduced hemoglobin is large.

The two light-emitting diodes 36 and 37 emit light in a time-sharing manner. The finger of an examined subject is irradiated with the light emitted by the light-emitting diodes 36 and 37 via the irradiating optical fibers 31 and 32. The light shone on the finger from the light-irradiating optical fiber 31 is reflected by the skin, and the reflected light is detected by the photodiode 38, the scattered light is detected by the photodiode 39, and the traveled photon is incident on the light-receiving optical fiber 33 and is then detected by the photodiode 40. The traveled photon-receiving optical fiber 33 is adapted to be in close contact with the finger surface such that it can avoid the direct entry of reflected and/or scattered light. The light with which the finger is irradiated via the light-irradiating optical fiber 32 is reflected by the skin of the finger, and the reflected light is detected by the photodiode 39, the scattered light is detected by the photodiode 38, and the traveled photon is incident on the light-receiving optical fiber 35 and is then detected by the photodiode 40. Thus, by causing the two light-emitting diodes 36 and 37 to emit light in a time-divided manner, the photodiodes 38 and 39 can detect different light depending on the irradiating position of the light-irradiating optical fibers. By this structure, the size of the optical sensor portion 18 can be reduced. With regard to the light shone on the finger via the light-irradiating fiber 32, the light-receiving optical fiber 33 may be adapted not to detect traveled photon.

By thus employing the structure such that the light reflected by the skin of the finger and the scattered light are received directly by photodiodes, the received amount of light detected by each photodiode can be increased. Regarding the light-receiving optical fiber 33, it is similarly possible to increase the amount of received light by disposing the photodiode 40 directly at the position corresponding to the end of the light-receiving optical fiber 33. However, putting the photodiode 40 directly at the end of the light-receiving optical fiber 33 would result in an increased size of the optical sensor portion 18. Accordingly, it is desirable to use the light-receiving optical fiber 33 if the size of the optical sensor portion 18 is to be further reduced.

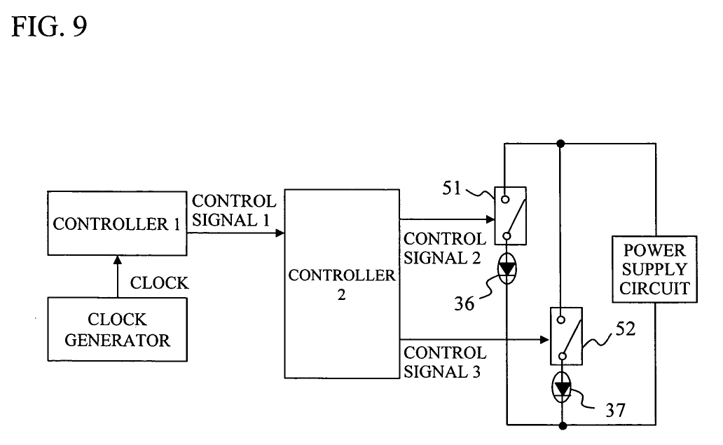

FIG. 9 shows a block diagram of an example of the circuit for causing the light-emitting diodes to emit in a time-divided manner. A controller 1 causes the light-emitting diodes 36 and 37 to emit light in a time-divided manner by repeating the following steps (1) and (2). FIG. 9 concerns the case of using two wavelengths (two LEDs).

(1) Sends a control signal 1 to a controller 2 in synchronism with the clock of a clock generator for a certain duration of time in order to select a control signal 2. As a result, a switching circuit 51 is turned on, thereby turning power on and causing the light-emitting diode 36 to emit light.

(2) After a certain duration of time has elapsed, sends a control signal 1 to the controller 2 in synchronism with the clock of the clock generator for a certain duration of time in order to select a control signal 3. As a result, a switching circuit 52 is turned on, thereby turning power on and causing the light-emitting diode 37 to emit light.

It is also possible to cause the two light-emitting diodes 36 and 37 to emit nearly simultaneously, rather than in a time-divided manner. In an example of a method of separately detecting light from a plurality of light sources, the individual light sources are driven by modulating them with different modulation frequencies. In this method, light from each light source can be separately detected by focusing on the frequency components contained in a detection signal from photodetectors.

The arrangement of the light-irradiating optical fibers, photodiodes, and the light-receiving optical fibers in the optical sensor portion 18 is determined based on the following theories (1) to (3).

(1) Regarding the position of the reflected-light receiving photodiode with respect to the light-irradiating optical fiber, it is most appropriate to position the light-receiving plane of the photodetector at a position where the reflected light is theoretically received, namely at a position within the plane of incidence of light on the subject where light reflected in a direction with an outgoing angle that is equal to the angle of incidence on a light-incident point of the subject is received. By locating the light-receiving plane of the reflected-light receiving photodiode at such a position, the ratio of reflected light in the amount of received light can be maximized.

(2) The scattered light-receiving photodetector has the light-receiving plane thereof disposed in a plane that forms an angle of approximately 90° with respect to the plane of incidence of light on the subject. The scattered-light receiving photodetector is disposed at approximately 90° relative to the reflected-light receiving photodetector because the source of light detected as scattered light is desired to be narrowed to the scattering phenomena as much as possible, as opposed to theory (1), or because the range of the phenomena as the object of detection of scattering is desired to be increased by the provision of the large angle of approximately 90°.

(3) The light-receiving end of the light-receiving optical fiber for traveled photon is disposed at a position in the plane of incidence of light on the subject that is farther than the light-receiving plane of the reflected-light receiving photodetector with respect to the light-irradiating optical fiber. The light-receiving end of the traveled-photon receiving optical fiber is thus disposed in the plane of incidence of light on the subject for the following reason. During the process in which light enters the skin and is scattered inside, the distribution of light spreads, and yet the distribution is greatest in the direction of incidence. As a result, the amount of light exiting from the skin is also greatest in this direction, so that the traveled photon can be most efficiently detected. Further, the light-receiving end of the traveled-photon receiving optical fiber is disposed farther than the light-receiving end of the reflected-light receiving photodetector with respect to the light-irradiating optical fiber. By so doing, a large amount of information can be detected, such as information relating to the absorption of light by hemoglobin in blood flowing in the capillary blood tubes during the process of light penetrating the skin and being scattered inside, or information relating to the thickness of skin, for example. It is also possible, however, to dispose the light-receiving optical fiber for traveled photon at a position other than that in the plane of incidence of light on the subject, though in that case the amount of traveled photon that is detected would be reduced.

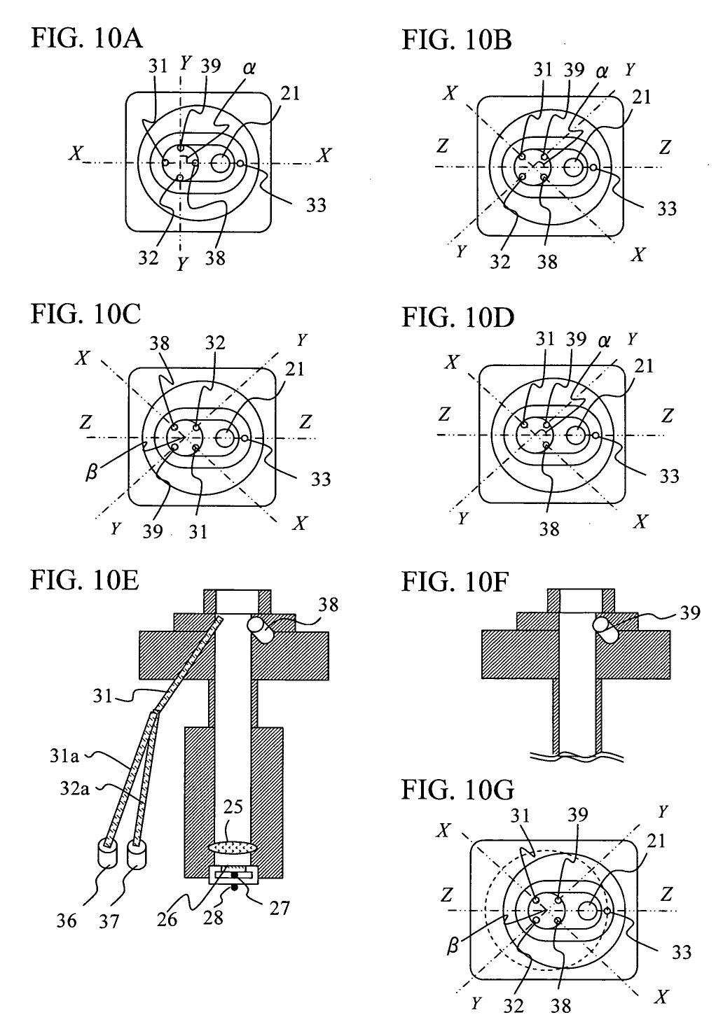

In accordance with those theories (1) to (3), the exiting ends of the light-irradiating optical fibers, the photodetectors, and the receiving end of the light-receiving optical fiber are disposed in the optical sensor portion 18 as shown in the plan view of FIG. 10A. In this plan view, the light-irradiating optical fiber 31, the reflected-light receiving photodiode 38 and the traveled-photon receiving optical fiber 33 are disposed substantially along an identical line XX. On a line YY with an angle α of approximately 90° with respect to the line XX connecting the light-irradiating optical fiber 31 and reflected-light receiving photodiode 38, there are disposed the light-irradiating optical fiber 32 and the scattered-light receiving photodiode 39 for receiving scattered light from the light-irradiating optical fiber 31. The light-irradiating optical fibers 31 and 32 and the photodiodes 38 and 39 are disposed more or less on an identical circle P about a center point at which the lines XX and YY intersect.

Regarding the angles of irradiation and detection of light by the light-irradiating optical fiber 31 and photodiode 38, the reflected-light receiving photodiode 38 is positioned such that it can receive a beam of light reflected at a point (light incident point on the subject) y, namely the point y in FIG. 8E, that is located above the point of intersection of the lines XX and YY shown in FIG. 10A with the same angle as an incident angle θ that is formed by the axis of the light-irradiating optical fiber 31 and a normal to the surface of the subject at the incident point γ when the light emitted by the light-irradiating optical fiber 31 is reflected at the light incident point γ. Namely, the light-irradiating optical fiber 31 and the photodiode 38 are disposed such that the angles θ and φ are substantially identical.

By thus disposing the light-irradiating optical fiber 31, the photodiode 38 and the traveled-photon receiving optical fiber 33 along the same line as shown in FIG. 10A, the amount of traveled photon detected by the light-receiving optical fiber 33 can be maximized. However, since the light-receiving optical fiber 33 is disposed in the same direction as the direction in which light is emitted from the light-irradiating optical fibers 31 and 32, the ratio of reflected light or scattered light in the amount of received light increases. Further, as the photodiode 38, the plate 21, and the heat-conducting member 22 and thermistor 24 connected thereto are disposed along an identical line, the plate 21 and the heat-conducting member 22 and thermistor 24 connected thereto must be disposed away from the light-receiving optical fiber 33 along the line XX in order to allow the photodiode 38 to be disposed. As a result, the size of the optical sensor portion shown in FIG. 10A increases.

Alternatively, the exiting ends of the light-irradiating optical fibers, the photodiodes, and the receiving end of the light-receiving optical fiber may be disposed in the optical sensor portion 18 as shown in a plan view of FIG. 10B, in accordance with the theories (1) to (3). In the plan view, the light-irradiating optical fiber 31 and the reflected-light receiving photodiode 38 are disposed along the identical line XX. On a line YY with an angle of approximately 90° with respect to the line XX connecting the light-irradiating optical fiber 31 and reflected-light receiving photodiode 38, there are disposed the light-irradiating optical fiber 32 and the light-receiving photodiode 39 for receiving scattered light from the light-irradiating optical fiber 31. The traveled-photon receiving optical fiber 33 is disposed along a line ZZ that intersects the line YY at an angle α of approximately 45°. The light-irradiating optical fibers 31 and 32 and the photodiodes 38 and 39 are disposed more or less on an identical circle P about a center where the lines XX and YY intersect. Regarding the angles of irradiation and detection of light by the light-irradiating optical fiber 31 and the photodiode 38, the reflected-light receiving photodiode 38 is positioned such that it can receive a beam of light reflected at a point (light incident point on the subject) γ, namely the point γ in FIG. 8E, that is located above the point of intersection of the lines XX and YY shown in FIG. 10A with the same angle as an incident angle θ that is formed by the axis of the light-irradiating optical fiber 31 and a normal to the surface of the subject at the incident point γ when the light emitted by the light-irradiating optical fiber 31 is reflected at the light incident point γ. Namely, the light-irradiating optical fiber 31 and the photodiode 38 are disposed such that the angles θ and +are substantially identical.

By thus disposing the traveled-photon receiving optical fiber 33 on the line ZZ as shown in FIG. 10B, though the amount of traveled photon that can be detected by the light-receiving optical fiber 33 decreases, the distance between the point of intersection of the lines XX and YY and the light-receiving optical fiber 33 can be reduced in the line ZZ direction. Accordingly, the size of the optical sensor portion 18 can be reduced. Further, as the light-receiving optical fiber 33 is disposed at a position approximately 45° away from the direction in which the light-irradiating optical fibers 31 and 32 radiate, the influence of reflected light or scattered light can be minimized, so that a large amount of traveled photon can be detected in the amount of received light.

Further alternatively, the exiting ends of the light-irradiating optical fibers, the photodiodes, and the receiving end of the light-receiving optical fiber in the optical sensor portion 18 may be disposed as shown in a plan view of FIG. 10C, in accordance with the theories (1) to (3). Namely, the light-irradiating optical fibers 31 and 32 and the photodiodes 38 and 39 may be disposed at any position on a circle P with a center β and a radius corresponding to the line between the center β and the light-irradiating optical fiber 31 as long as the relationship of the line XX intersecting the line YY at approximately 90° is maintained. For example, as shown in FIG. 10C, the optical sensor portion 18 may be configured in the following manner. The light-irradiating optical fiber 31 is disposed at a position corresponding to that of the photodiode 38 of FIG. 10B, and the photodiode 38 is disposed at a position corresponding to that of the light-irradiating optical fiber 31 of FIG. 10B. The light-irradiating optical fiber 32 is disposed at a position corresponding to that of the photodiode 39 of FIG. 10B, and the photodiode 39 is disposed at a position corresponding to that of the light-irradiating optical fiber 32 of FIG. 10B. The traveled-photon receiving optical fiber 33 is disposed on the line ZZ that intersects the line YY at approximately 45°. Regarding the angles of irradiation and detection of light by the light-irradiating optical fiber 31 and the photodiode 38, the reflected-light receiving photodiode 38 is positioned such that it can receive a beam of light reflected at a point (light incident point on the subject) γ, namely the point γ in FIG. 8E, that is located above the point of intersection of the lines XX and YY shown in FIG. 10C with the same angle as an incident angle θ that is formed by the axis of the light-irradiating optical fiber 31 and a normal to the surface of the subject at the incident point γ when the light emitted by the light-irradiating optical fiber 31 is reflected at the light incident point γ. Namely, the light-irradiating optical fiber 31 and the photodiode 38 are disposed such that the angles θ and φ are substantially identical.

In this arrangement, the traveled-photon receiving optical fiber 33 is positioned in a direction opposite to that in which the light-irradiating optical fibers 31 and 32 radiate, so that, although the amount of light received by the light-receiving optical fiber 33 is fairly small, the received light contains hardly any reflected light or scattered light and consists mostly of traveled photon.

Regarding the arrangement of the light-irradiating optical fibers, photodiodes, and light-receiving optical fiber in the optical sensor portion 18 shown in FIGS. 10A to 10C, a branch optical fiber 32a may be connected to the light-irradiating optical fiber 31, and a light-emitting diode 37 may be disposed at the end of the optical fiber 32a, instead of using the light-irradiating optical fiber 32. A top view of this arrangement of the optical fibers and the light-emitting diode is shown in FIG. 10D. FIG. 10E is a cross section taken along line XX of FIG. 10D, and FIG. 10F a cross section taken along line YY. The ZZ cross section of FIG. 10D is similar to that of FIG. 8B.

Regarding the optical sensor portion 18, the exiting ends and the receiving planes of the light-irradiating optical fibers 31 and 32 and the photodiodes 38 and 39, respectively, may be displaced a little in their optical axial directions as long as they are aimed at the light incident point γ on the subject (see FIG. 8E). In that case, the light-irradiating optical fibers 31 and 32 and the photodiodes 38 and 39 would not be all disposed on the identical circle P as shown, but would be displaced from one another in the vertical or height direction. However, if the light-irradiating optical fibers 31 and 32 are disposed at different heights, the intensity of irradiated light would be larger near the body surface and would be lower away from the body surface. Further, if the photodiodes 38 and 39 are disposed at different heights, the intensity of detected light would increase near the body surface and would decrease away from the body surface due to the spreading of light. Thus, such an arrangement would make it difficult to carry out measurement in a uniform environment and a correction of the information detected by the photodiodes would be necessary. In general, the light-irradiating optical fibers and the photodiodes are disposed near where light is irradiated so that an accurate measurement can be conducted. In the configuration of the present invention, the light-irradiating optical fibers and the photodiodes are disposed as close to the body surface as possible without hindering other functions for measurements, such as the measurement of temperatures. Further, the light-irradiating optical fibers 31 and 32 and the photodiodes 38 and 39 are disposed on the identical circle P or, more generally, near the plane of a cone whose apex is at the point y of incidence of light on the subject, such that a uniform environment for measuring radiated light and detected light can be obtained and an accurate measurement can be conducted.

Further regarding the optical sensor portion 18 shown in FIGS. 10A to 10D, the traveled-photon receiving optical fiber 33 may be disposed at any point on a circle with a center β and a radius corresponding to the line connecting the center β and the light-receiving optical fiber 33, as indicated by a dashed line in FIG. 10G. In this case, the distance between the exiting end (the light incident point) of the light-irradiating optical fiber and the receiving end of the traveled-photon receiving optical fiber 33 (where the light as the object of reception is incident) would be larger than the distance between the light incident point and the reception end of the photodiode 38 or the receiving end of the photodiode 39. In such an arrangement, the placement of the traveled-photon receiving optical fiber 33 can be freely set, so that the optical sensor portion 18 can be configured in various ways as needed.

The photodiodes 38 and 39 provide reflectance R as measurement data, and absorbance can be approximately calculated from log(1/R). Light of wavelengths 810 nm and 950 nm is irradiated, and R is measured for each and log(1/R) is obtained for each, so that absorbance A_{D11} and A_{D21} at wavelength 810 nm and absorbance A_{D12} and A_{D22} at wavelength 950 nm can be measured. Part of the light penetrates into the skin and travels a certain distance d while being scattered therein repeatedly. The intensity I_{D3i} of traveled photon is measured by a photodiode 40. (The absorbance of reflected light of wavelength λᵢ detected by the photodiode for detecting reflected light is referenced by A_{D1i}, the absorbance of scattered light of wavelength λᵢ detected by the photodiode for detecting scattered light is referenced by A_{D2i}, and the intensity of traveled photon of wavelength λᵢ detected by the photodiode 40 is referenced by I_{D3i}.)

When the reduced hemoglobin concentration is [Hb] and the oxyhemoglobin concentration is [HbO₂], scattered-light absorbance A_{D2i} at wavelength λᵢ is expressed by the following equations:

$${A}_{D2i} = a \{ [ Hb ] \times {A}_{Hb} ( λ i ) + [ {Hb O}_{2} ] \times {A}_{{HbO}_{2}} ( λ i ) \} \times D \times {a}_{R}$$

$${a}_{R} = \frac{b \times \sum {A}_{D 2 i}}{\sum {A}_{D 1 i}} , D = \frac{1}{\frac{c \times \sum {I}_{D 3 i}}{i}}$$

where A_{Hb}(λᵢ) and A_{Hb02}(λᵢ) are the molar absorbance coefficients of the reduced hemoglobin and the oxyhemoglobin, respectively, and are known at the respective wavelengths. Terms a, b, and c are proportionality coefficients. A_{D1i} is the reflected-light absorbance at wavelength λᵢ, and I_{D3i} is the traveled photon intensity at wavelength λᵢ. From the above equations, the parameter a_{R}, which is determined by the relationship between reflected light and scattered light, and the parameter D of the skin thickness can be determined as constants, and can be substituted in the equation of A_{D2i}. The parameter determined by the relationship between reflected light and scattered light is a parameter relating to the roughness of the skin surface, for example, and the influence of the roughness of the skin surface, for example, can be corrected using that parameter. The parameter relating to the thickness of the skin can be determined from the measurement value obtained by the traveled-photon detector, and the influence of the thickness of the skin can be corrected using that parameter. Since i=2 wavelengths, two equations of A_{D2i} are produced. By solving these simultaneous equations, the two variables to be obtained, namely [Hb] and [HbO₂], can be obtained. The hemoglobin concentration [Hb]+[HbO₂], and the hemoglobin oxygen saturation [HbO₂]/([Hb]+[HbO₂]) can be determined from the above-obtained [Hb] and [HbO₂].

Although the present example has been described with regard to the measurement of the hemoglobin concentration and the hemoglobin oxygen saturation based on the measurement of absorbance at two wavelengths, absorbance may be measured by adding one or more wavelengths at which the difference in molar absorbance coefficient between the oxyhemoglobin and the deoxyhemoglobin is large so as to increase the measurement accuracy.

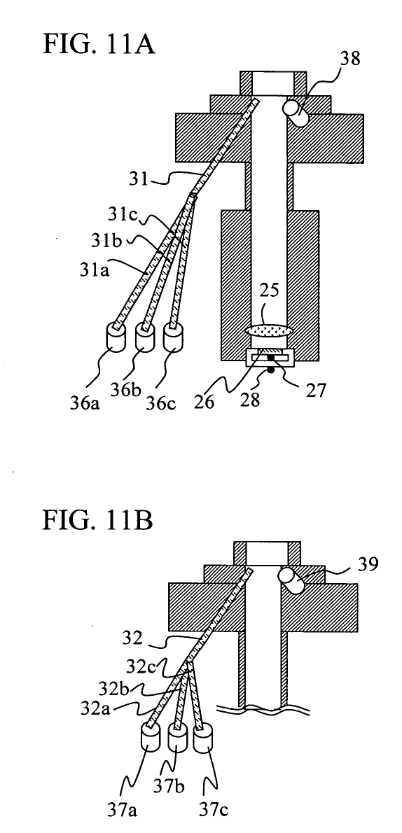

For example, when six wavelengths are used for measurement, any of the configurations shown in FIGS. 10A to 10C may be employed for the arrangement of the light-irradiating optical fibers, photodiodes, and the light-receiving optical fiber in the optical sensor portion 18 in accordance with the theories (1) to (3). However, in the case of six wavelengths, while the ZZ cross-section of FIG. 10B corresponds to FIG. 8B, the XX cross-section of FIG. 10B corresponds to FIG. 11A, and the YY cross-section of FIG. 10B corresponds to FIG. 11B. To the light-irradiating optical fiber 31 are connected three branch optical fibers 31a, 31b and 31c each provided at the end thereof with light-emitting diodes 36a, 36b and 36c, respectively. Likewise, three branch optical fibers 32a, 32b and 32c are connected to the light-irradiating optical fiber 32, and light-emitting diodes 37a, 37b and 37c are connected to the ends of the respective branch optical fibers. By thus connecting three branch optical fibers to one light-irradiating optical fiber in a bundled manner, the size of the optical sensor portion 18 can be reduced.

The light-emitting diode 36a emits light of 810 nm, light-emitting diode 36b light of 880 nm, light-emitting diode 36c light of 950 nm, light-emitting diode 37a light of 450 nm, light-emitting diode 37b light of 520 nm, and light-emitting diode 37c light of 660 nm, for example. Using the result of detection of irradiated light having these six wavelengths, corrections can be made for the influences of interfering components on the determination of hemoglobin concentration and hemoglobin oxygen saturation from absorbance, the interfering components including melanin pigment, bilirubin and the turbidity of blood, for example. Thus, the accuracy of measurement can be improved.

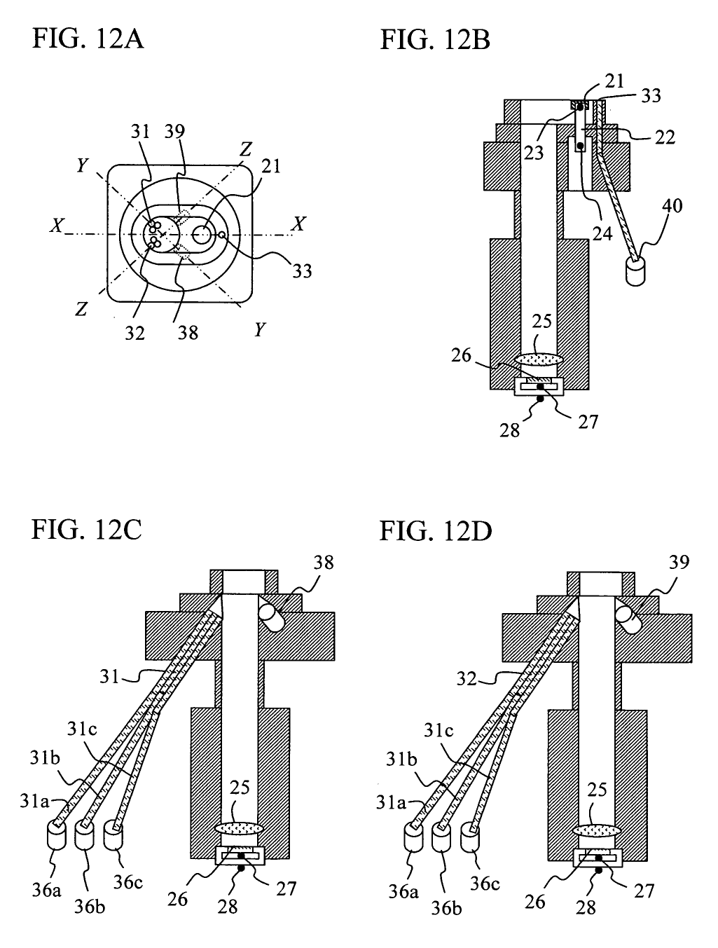

As mentioned above, by providing the light-irradiating optical fiber with three branches, an ideal configuration can be obtained in which the three light-emitting diodes share the same point of light irradiation. However, since the actual fiber-irradiated light has certain spread, the same function can be provided by employing a structure in which a light-irradiating fiber is provided to each light-emitting diode and the tips of the fibers are bundled. This structure, which can employ conventional fibers and can therefore be made inexpensively, is shown in FIGS. 12A to 12D. FIG. 12A shows a front view, FIG. 12B shows an XX cross-section, FIG. 12C shows a YY cross-section, and FIG. 12D shows a ZZ cross-section. As shown in FIG. 12A, a light-irradiating optical fiber 31 consists of three fibers bundled at the tips thereof. As shown in FIG. 12B, a photodiode 40 is disposed using a fiber 33 in order to allow for an efficient reception of traveled photon. As shown in FIG. 12C, optical fibers 31 are connected to light-irradiating light-emitting diodes 36, and the tips of the fibers are bundled together, such that each optical fiber 31 emits light from a corresponding light-emitting diode. The photodiode 38 may be disposed at a further spaced-apart position if its housing has a lens function. FIG. 12D shows a similar configuration.

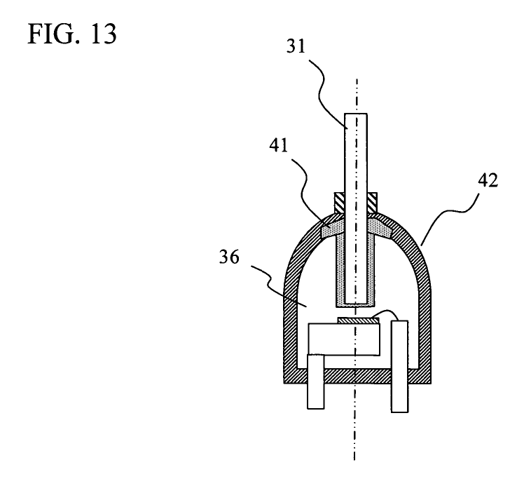

FIG. 13 shows an example of connection and blocking of light between an light-emitting diode and an optical fiber. A light-emitting diode 36 is provided with an opening with a slightly larger diameter than the external diameter of an optical fiber 31. The opening is filled with an adhesive 41 and then the optical fiber 31 is inserted and fixed therein such that the light emitted by the light-emitting diode 36 can be guided in the direction opposite to the face of the optical fiber. The opening also functions to position and fix the optical fiber 31 in place. It goes without saying that the intensity of irradiation increases as the depth of the opening is closer to the plane of the light-emitting diode element. The adhesive 41 should preferably be one that absorbs only a little amount of the irradiated light and have a refractive index that is close to that of the light-emitting diode and the optical fiber core. The light-emitting diode 37 and the traveled-photon receiving photodiode 40 have the same structures.

In practice, the light-emitting diodes 36 and 37 and the photodiode 40 are equipped with a light-blocking cap 42 for preventing the leakage of light to the outside and the reception of light from the outside. The light-blocking cap 42 is formed by a soft material, such as a silicon resin, so that it can be easily mounted on the light-emitting diode or optical fiber assembly. With regard to the photodiodes 38 and 39, which are disposed inside the sensor portion, there might be no need to provide such a measure because they are disposed inside the sensor portion and are therefore already blocked against external light.

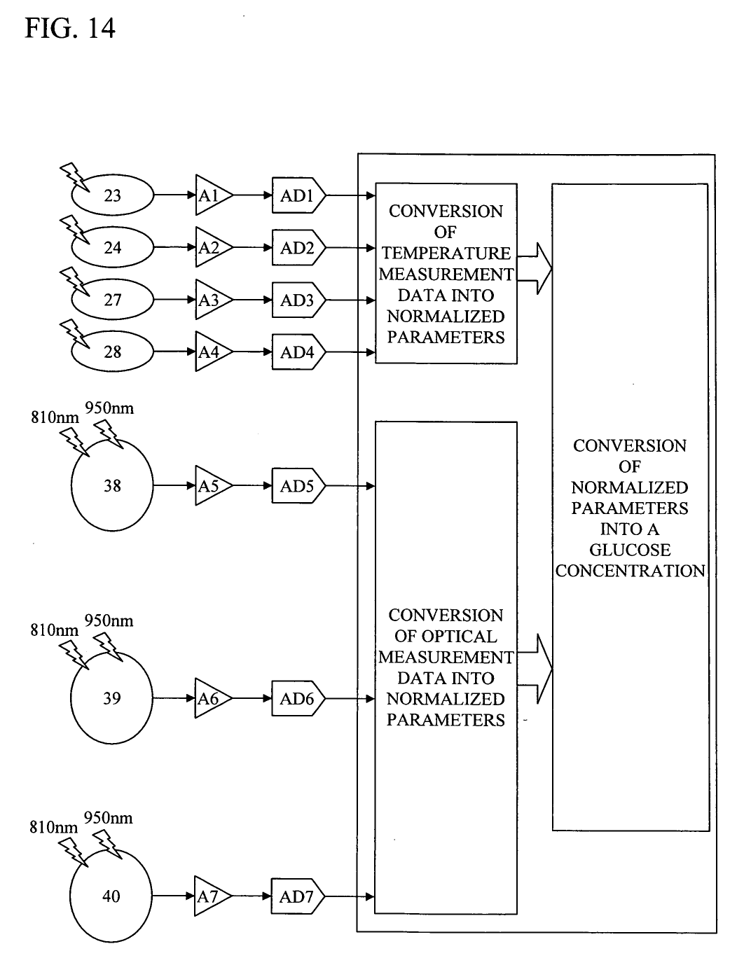

FIG. 14 is a conceptual chart showing the flow of data processing in the apparatus using two wavelengths. The apparatus according to the present example is equipped with a thermistor 23, a thermistor 24, a pyroelectric detector 27, a thermistor 28, and three photodetectors formed by photodiodes 38 to 40. The photodiodes 38 and 39 measure absorbance at wavelengths 810 nm and 950 nm. The photodiode 40 measures the intensity at wavelengths 810 nm and 950 nm. Thus, the apparatus is supplied with ten kinds of measurement values including temperature, thermal, and optical measurement data. In the case where the wavelength 880 nm is added for improving accuracy, the number of kinds of measurement values fed to the apparatus would be 13.

The seven kinds of analog signals are supplied via individual amplifiers A1 to A7 to analog/digital converters AD1 to AD7, where they are converted into digital signals. Based on the digitally converted values, parameters xᵢ (i=1, 2, 3, 4, 5) are calculated. The following are specific descriptions of xᵢ (where e₁ to e₅ are proportionality coefficients):

Parameter proportional to heat radiation
x₁=e₁×(T₃)⁴

Parameter proportional to heat convection
x₂=e₂×(T₄−T₃)

Parameter proportional to hemoglobin concentration
x₃=e₃×([Hb]+[Hb₂])

Parameter proportional to hemoglobin saturation

$${x}_{4} = {e}_{4} \times ( \frac{[ {HbO}_{2} ]}{[ Hb ] + [ {HbO}_{2} ]} )$$

Parameter proportional to blood flow volume

$${x}_{5} = {e}_{5} \times ( \frac{1}{{t}_{CONT} \times ( {S}_{1} - {S}_{2} )} )$$

Then, normalized parameters are calculated from mean values and standard deviations of parameter xᵢ obtained from actual data pertaining to large numbers of able-bodied people and diabetic patients. A normalized parameter Xᵢ (where i=1, 2, 3, 4, 5) is calculated from each parameter xᵢ according to the following equation:

$${X}_{i} = \frac{{x}_{i} - {\overline{x}}_{i}}{SD ( {x}_{i} )}$$

where

xᵢ: parameter

\overline{x}ᵢ: mean value of the parameter

SD(xᵢ): standard deviation of the parameter

Using the above five normalized parameters, calculations are conducted for conversion into a glucose concentration to be eventually displayed. A program necessary for the processing calculations is stored in a ROM in the microprocessor built inside the apparatus. The memory area required for the processing calculations is secured in a RAM similarly built inside the apparatus. The results of calculation are displayed on the LCD.

The ROM stores, as a constituent element of the program necessary for the processing calculations, a function for determining glucose concentration C in particular. The function is defined as follows. C is expressed by the below-indicated equation (1), where aᵢ (i=0, 1, 2, 3, 4, 5) is determined from a plurality of pieces of measurement data in advance according to the following procedure:

(1) A multiple regression equation is created that indicates the relationship between the normalized parameters and the glucose concentration C.

(2) Normalized equations (simultaneous equations) relating to the normalized parameters are obtained from equations obtained by the least-squares method.

(3) Values of coefficient aᵢ (i=0, 1, 2, 3, 4, 5) are determined from the normalized equations and then substituted into the multiple regression equation.

Initially, the regression equation (1) indicating the relationship between the glucose concentration C and the normalized parameters X₁, X₂, X₃, X₄, and X₅ is formulated.

$$\begin{matrix}C = f ( {X}_{1} , {X}_{2} , {X}_{3} , {X}_{4} , {X}_{5} ) \\ = {a}_{0} + {a}_{1} {X}_{1} + {a}_{2} {X}_{2} + {a}_{3} {X}_{3} + {a}_{4} {X}_{4} + {a}_{5} {X}_{5}\end{matrix} \qquad ( 1 )$$

Then, the least-squares method is employed to obtain a multiple regression equation that would minimize the error with respect to a measured value Cᵢ of glucose concentration according to an enzyme electrode method. When the sum of squares of the residual is E, E is expressed by the following equation (2):

$$\begin{matrix}E = {\sum }_{i = 1}^{n} {d}_{i}^{2} \\ = {\sum }_{i = 1}^{n} {( {C}_{i} - f ( {X}_{i 1} , {X}_{i 2} , {X}_{i 3} , {X}_{i 4} , {X}_{i 5} ) )}^{2} \\ = {\sum }_{i = 1}^{N} {\{ {C}_{i} - ( {a}_{0} + {a}_{1} {X}_{i 1} + {a}_{2} {X}_{i 2} + {a}_{3} {X}_{i 3} + {a}_{4} {X}_{i 4} + {a}_{5} {X}_{i 5} ) \}}^{2}\end{matrix} \qquad ( 2 )$$

The sum E of squares of the residual becomes minimum when partial differentiation of equation (2) with respect to a₀, a2, . . . , a₅ gives zero. Thus, we have the following equations:

$$\frac{\partial E}{\partial {a}_{0}} = - 2 {\sum }_{i = 1}^{n} \{ {C}_{i} - ( {a}_{0} + {a}_{1} {X}_{i 1} + {a}_{2} {X}_{i 2} + {a}_{3} {X}_{i 3} + {a}_{4} {X}_{i 4} + {a}_{5} {X}_{i 5} ) \} = 0 \\ \frac{\partial E}{\partial {a}_{1}} = - 2 {\sum }_{i = 1}^{n} {X}_{i 1} \{ {C}_{i} - ( {a}_{0} + {a}_{1} {X}_{i 1} + {a}_{2} {X}_{i 2} + {a}_{3} {X}_{i 3} + {a}_{4} {X}_{i 4} + {a}_{5} {X}_{i 5} ) \} = 0 \\ \frac{\partial E}{\partial {a}_{2}} = - 2 {\sum }_{i = 1}^{n} {X}_{i 2} \{ {C}_{i} - ( {a}_{0} + {a}_{1} {X}_{i 1} + {a}_{2} {X}_{i 2} + {a}_{3} {X}_{i 3} + {a}_{4} {X}_{i 4} + {a}_{5} {X}_{i 5} ) \} = 0 \\ \frac{\partial E}{\partial {a}_{3}} = - 2 {\sum }_{i = 1}^{n} {X}_{i 3} \{ {C}_{i} - ( {a}_{0} + {a}_{1} {X}_{i 1} + {a}_{2} {X}_{i 2} + {a}_{3} {X}_{i 3} + {a}_{4} {X}_{i 4} + {a}_{5} {X}_{i 5} ) \} = 0 \\ \frac{\partial E}{\partial {a}_{4}} = - 2 {\sum }_{i = 1}^{n} {X}_{i 4} \{ {C}_{i} - ( {a}_{0} + {a}_{1} {X}_{i 1} + {a}_{2} {X}_{i 2} + {a}_{3} {X}_{i 3} + {a}_{4} {X}_{i 4} + {a}_{5} {X}_{i 5} ) \} = 0 \\ \frac{\partial E}{\partial {a}_{5}} = - 2 {\sum }_{i = 1}^{n} {X}_{i 5} \{ {C}_{i} - ( {a}_{0} + {a}_{1} {X}_{i 1} + {a}_{2} {X}_{i 2} + {a}_{3} {X}_{i 3} + {a}_{4} {X}_{i 4} + {a}_{5} {X}_{i 5} ) \} = 0 \\ \qquad ( 3 )$$

When the mean values of C and X₁ to X₅ are Cₘₑₐₙ and X₁ₘₑₐₙ to X₅ₘₑₐₙ, respectively, since Xᵢₘₑₐₙ=0 (i=1 to 5), equation (1) yields:

$$\begin{matrix}{a}_{0} = {C}_{mean} - {a}_{1} {X}_{1 mean} - {a}_{2} {X}_{2 mean} - {a}_{3} {X}_{3 mean} - {a}_{4} {X}_{4 mean} - {a}_{5} {X}_{5 mean} \\ = {C}_{mean}\end{matrix} \qquad ( 4 )$$

The variation and covariation between the normalized parameters are expressed by equation (5). Covariation between the normalized parameter Xᵢ (i=1 to 5) and C is expressed by equation (6).

$$\begin{matrix}{S}_{ij} = {\sum }_{k = 1}^{n} ( {X}_{ki} - {X}_{imean} ) ( {X}_{kj} - {X}_{jmean} ) = {\sum }_{k = 1}^{n} {X}_{ki} {X}_{kj} ( i , j = 1 , 2 , … 5 ) & ( 5 ) \\ {S}_{iC} = {\sum }_{k = 1}^{n} ( {X}_{ki} - {X}_{imean} ) ( {C}_{k} - {C}_{mean} ) = {\sum }_{k = 1}^{n} {X}_{ki} ( {C}_{k} - {C}_{mean} ) ( i = 1 , 2 , … 5 ) & ( 6 )\end{matrix}$$

Substituting equations (4), (5), and (6) into equation (3) and rearranging yields a set of simultaneous equations (normalized equations) (7). Solving the set of equations (7) yields a₁ to a₅.
a₁S₁₁+a₂S₁₂+a₃S₁₃+a₄S₁₄+a₅S₁₅=S_{1C}
a₁S₂₁+a₂S₂₂+a₃S₂₃+a₄S₂₄+a₅S₂₅=S_{2C}
a₁S₃₁+a₂S₃₂+a₃S₃₃+a₄S₃₄+a₅S₃₅=S_{3C}
a₁S₄₁+a₂S₄₂+a₃S₄₃+a₄S₄₄+a₅S₄₅=S_{4C}
a₁S₅₁+a₂S₅₂+a₃S₅₃+a₄S₅₄+a₅S₅₅=S_{5C} (7)

Constant term a₀ is obtained by means of equation (4). The thus obtained aᵢ (i=0, 1, 2, 3, 4, 5) is stored in ROM at the time of manufacture of the apparatus. In actual measurement using the apparatus, the normalized parameters X₁ to X₅ obtained from the measured values are substituted into regression equation (1) to calculate the glucose concentration C.

Hereafter, an example of the process of calculating parameter Xi will be described. The example concerns measurement values obtained from able-bodied persons. Coefficients for the parameter calculation equations are determined by temperature data and optical measurement data that have been measured in advance. The ROM in the microprocessor stores the following formula for the calculation of the parameter:

$$\begin{matrix}{x}_{1} = 0.98 \times {10}^{- 3} \times {( {T}_{3} )}^{4} \\ {x}_{2} = - 1.24 \times ( {T}_{4} - {T}_{3} ) \\ {x}_{3} = 1.36 \times ( [ Hb ] + [ {HbO}_{2} ] ) \\ {x}_{4} = 2.67 \times ( \frac{[ {HbO}_{2} ]}{[ Hb ] + [ {HbO}_{2} ]} ) \\ {x}_{5} = 1.52 \times {10}^{6} \times ( \frac{1}{{t}_{CONT} \times ( {S}_{1} - {S}_{2} )} )\end{matrix}$$

When T₃=36.5° C. is substituted in the above equations as a measurement value, for example, x₁=1.74×10³. When T₄=19.7° C. is substituted in the above equations, x₂=2.08×10. Then, before finding X₃, it is necessary to find [Hb] and [HbO₂]. The coefficients for a concentration calculation formula are determined by the scattered-light absorbance coefficient of each substance that has been measured in advance. Using that formula, [Hb] and [HbO₂] can be determined by solving the following set of simultaneous equations in the case of measurement using two wavelengths:

$$\begin{matrix}{A}_{D2\_ 810} = 1.86 \\ = 0.87 \{ 800 \times [ Hb ] + 1050 \times [ {HbO}_{2} ] \} \times 1.04 \times 0.85 \\ {A}_{D2\_ 950} = 2.02 \\ = 0.87 \{ 750 \times [ Hb ] + 1150 \times [ {HbO}_{2} ] \} \times 1.04 \times 0.85 \\ {a}_{R} = 0.85 \\ = \frac{1.35 \times ( 1.67 + 1.98 )}{( 2.65 + 3.14 )} \\ D = 1.04 \\ = \frac{1}{\frac{0.95 \times ( 1.02 + 1.01 )}{2}}\end{matrix}$$

Solving this set of simultaneous equations gives [Hb]=0.17 mmol/L and [HbO₂]=2.17 mmol/L. Thus we have x₃=3.18 and x_{4=2.48}. Then, substituting S₁=1.76×10², S₂=1.89×10, and t_{CONT}=²² seconds gives x₅=4.40×10².

The hemoglobin concentration ([Hb]+[HbO₂]) was calculated to be 2.34 mmol/L. When the hemoglobin concentration was measured at the same time by an invasive method, i.e. by blood sampling, the value was 2.28 mmol/L.

When the traveled photon is not similarly detected by the light-receiving optical fiber 33 at the same time, the information about the parameter of the thickness of the skin would not be obtained. In that case, the below-indicated simultaneous equations would be obtained, and solving them would yield [Hb]=0.18 mmol/L and [HbO₂]=2.26 mmol/L. Thus, the hemoglobin concentration ([Hb]+[HbO₂]) would be 2.44 mmol/L.

$$\begin{matrix}{A}_{D2\_ 810} = 1.86 \\ = 0.87 \{ 800 \times [ Hb ] + 1050 \times [ {HbO}_{2} ] \} \times 0.85 \\ {A}_{D2\_ 950} = 2.02 \\ = 0.87 \{ 750 \times [ Hb ] + 1150 \times [ {HbO}_{2} ] \} \times 0.85 \\ {a}_{R} = 0.85 \\ = \frac{1.35 \times ( 1.67 + 1.98 )}{( 2.65 + 3.14 )}\end{matrix}$$

Thus, it has been confirmed that the result of calculation in the case where traveled photon is detected by the light-receiving optical fiber 33 is closer to the value of hemoglobin concentration measured by blood sampling than the calculation result in the case where traveled photon is not detected by the light-receiving optical fiber 33. Thus, it has been shown that the measurement accuracy can be improved by providing the optical sensor portion 18 with the light-receiving optical fiber 33.

Next, X₁ to X₅ are obtained. X₁ to X₅ are the results of normalization of the above-obtained parameters X₁ to X₅. Assuming the distribution of a parameter is normal, 95% of a normalized parameter takes on values between −2 and +2. The normalized parameters can be determined by the following equations:

$$\begin{matrix}{X}_{1} = - 0.06 = \frac{1.74 \times {10}^{3} - 1.75 \times {10}^{3}}{167} \\ {X}_{2} = 0.04 = \frac{2.08 \times 10 - 2.06 \times 10}{5} \\ {X}_{3} = 0.05 = \frac{3.18 - 3.15}{0.60} \\ {X}_{4} = - 0.12 = \frac{2.48 - 2.54}{0.50} \\ {X}_{5} = 0.10 = \frac{4.40 \times {10}^{2} - 4.28 \times {10}^{2}}{120}\end{matrix}$$

From the above equations, we have normalized parameters X₁=−0.06, X₂=+0.04, X3=+0.05, X₄=−0.12, and X₅=+0.10.

Hereafter, an example of the process of calculating the glucose concentration will be described. The coefficients for regression equation (1) are determined in advance based on many items of data obtained from able-bodied persons and diabetics, and the ROM in the microprocessor stores the following formula for calculating the glucose concentration:
C=99.1+18.3×X₁−20.2×X₂−24.4×X₃−21.8×X₄−25.9×X₅

Substituting X₁ to X₅ into the above equation gives C=96 mg/dl. Substituting normalized parameters X₁=+1.15, X₂=−1.02, X₃=−0.83, X₄=−0.91, and X₅=−1.24, which can be obtained as an example of the measured values for a diabetic patient, in the equation yields C=213 mg/dl.

The following describes the results of measurement by the conventional enzymatic electrode method in which a blood sample is reacted with a reagent and the amount of resultant electrons is measured to determine glucose concentration, and the results of measurement by an embodiment of the invention. When the glucose concentration for an able-bodied person was 89 mg/dl according to the enzymatic electrode method in one example, substituting the normalized parameters X₁=−0.06, X₂=+0.04, X₃=+0.07, X₄=−0.10, and X₅=+0.10, which were obtained by measurement at the same time according to the invention, into the above equation yields C=95 mg/dl. In another example, when the measured value of glucose concentration for a diabetic patient was 238 mg/dl according to the enzymatic electrode method, substituting the normalized parameters X₁=+1.15, X₂=−1.02, X₃=−0.86, X₄=1.02, and X₅=−1.24, which were obtained by measurement at the same time according to the invention, into the above equation yields C=216 mg/dl. The results thus indicated that the method according to the invention can provide highly accurate glucose concentration values.

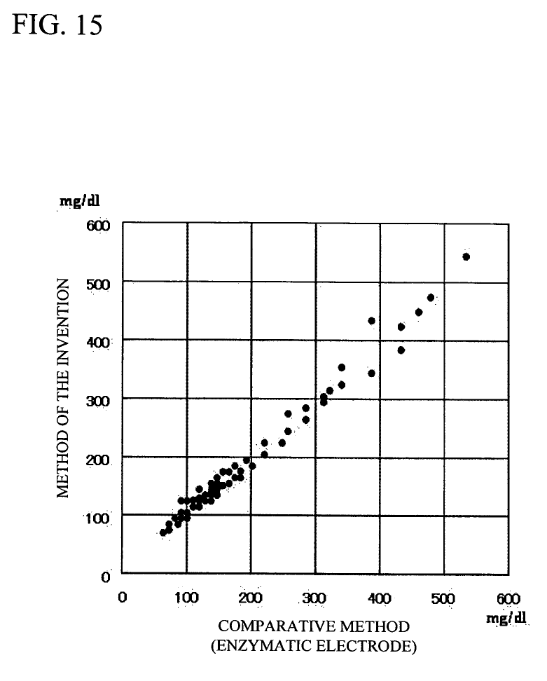

FIG. 15 shows a graph plotting glucose concentrations for a plurality of patients, with the vertical axis showing the calculated values of glucose concentration according to the invention, and the horizontal axis showing the measured values of glucose concentration according to the enzymatic electrode method. It is seen that a good correlation is obtained by measuring the oxygen supply volume and the blood flow volume according to the method of the invention (correlation coefficient=0.9394).

Thus, the invention makes it possible to determine blood sugar levels in a non-invasive measurement with similar levels of accuracy to the conventional invasive method.

All publication, patents, and patent applications cited herein are incorporated herein by reference to their entirety.

What is claimed is:

1. An optical measurement apparatus comprising:
a first light source for producing light of a first wavelength;
a first optical fiber for irradiating a light incident point on the surface of a subject with the light from said first light source;
a second light source for producing light of a second wavelength;
a second optical fiber for irradiating said light incident point on the surface of the subject with the light from said second light source in a direction different from that of the light of said first wavelength;
a first photodetector on which reflected light of the light of said first wavelength reflected by said light incident point and scattered light of the light of said second wavelength are incident without via fiber;
a second photodetector on which reflected light of the light of said second wavelength reflected at said light incident point and scattered light of the light of said first wavelength are incident without via fiber;
a third photodetector; and
a third optical fiber having an incident end thereof disposed at such a position as to be in contact with the surface of said subject, said third optical fiber being adapted to receive light exiting from an area spaced apart from said light incident point on the surface of said subject and then transmit the light to said third detector.

2. The optical measurement apparatus according to claim 1, wherein
said first and second light sources are adapted to emit light in a time-divided manner so that said light incident point on the surface of said subject is irradiated with the light of said first wavelength and the light of said second wavelength in a time-divided manner;
the reflected light of said first wavelength from said light incident point is mainly incident on said first photodetector when said first light source is emitting, while the scattered light of said second wavelength is mainly incident when said second light source is emitting; and
the reflected light of said second wavelength from said light incident point is mainly incident on said second photodetector when said second light source is emitting, and the scattered light of said first wavelength is mainly incident when said first light source is emitting.

3. The optical measurement apparatus according to claim 1, wherein a plane of incidence of the light of said first wavelength and a plane of incidence of the light of said second wavelength are substantially perpendicular to each other with respect to said light incident point on the surface of said subject.

4. The optical measurement apparatus according to claim 1, wherein an exiting end of said first optical fiber, an exiting end of said second optical fiber, a receiving plane of said first photodetector, and a receiving plane of said second photodetector are disposed in the vicinity of a circular conical surface with an apex thereof located at said light incident point on the surface of said subject.

5. The optical measurement apparatus according to claim 1, wherein the distance between said light incident point on the surface of said subject and the incident end of said third optical fiber is larger than the distance between the light incident point and the incident plane of said first photodetector and the receiving plane of said second photodetector.

6. The optical measurement apparatus according to claim 1, wherein the incident end of said third optical fiber is located on the plane of incidence of the light of said first wavelength or on the plane of incidence of the light of said second wavelength.

7. The optical measurement apparatus according to claim 1, wherein said first wavelength is a wavelength at which the molar absorption coefficient of oxyhemoglobin and that of reduced hemoglobin are equal, and said second wavelength is a wavelength used for the detection of a difference in absorbance between oxyhemoglobin and reduced hemoglobin.

8. The optical measurement apparatus according to claim 1, wherein a measurement error due to the thickness of skin is corrected using optical intensity measured by said third detector.

9. The optical measurement apparatus according to claim 1, wherein a branch optical fiber is connected to said first optical fiber and/or said second optical fiber, and a light source for emitting light of a wavelength different from that of said first light source and said second light source is provided at an end of said branch optical fiber.

10. The optical measurement apparatus according to claim 1, wherein reflected light of said first wavelength reflected at said light incident point and scattered light of said second wavelength are directly incident on said first photodetector, and reflected light of said second wavelength reflected at said light incident point and scattered light of said first wavelength are directly incident on said second photodetector.

11. A blood sugar level measuring apparatus comprising:
(1) a heat-amount measuring portion for measuring a plurality of temperatures deriving from a body surface and acquiring information that is used for calculating a convective heat transfer amount and radiation heat transfer amount that are related to the dissipation of heat from the body surface;
(2) a blood flow volume measuring portion for acquiring information about the volume of blood flow;
(3) an optical measurement portion including a light source for producing light of at least two different wavelengths, an optical system for irradiating the body surface with the light emitted by said light source, and at least three different photodetectors for detecting the light that has been irradiated onto the body surface, said optical measurement portion providing hemoglobin concentration and hemoglobin oxygen saturation in blood;
(4) a memory portion in which relationships between parameters respectively corresponding to said plurality of temperatures, blood flow volume, hemoglobin concentration and hemoglobin oxygen saturation in blood, and blood sugar levels are stored;
(5) a calculation portion for converting a plurality of measurement values inputted from said heat amount measuring portion, said blood flow volume measuring portion, and said optical measurement portion respectively into said parameters, and then calculating a blood sugar value by applying said parameters to said relationships stored in said memory portion; and
(6) a display portion for displaying the blood sugar level calculated by said calculation portion, wherein
said optical measurement portion includes a first light source for producing light of a first wavelength, a second light source for producing light of a second wavelength, a first optical fiber, a second optical fiber, a third optical fiber, a first photodetector, a second photodetector, and a third photodetector,
a light incident point on the surface of a subject is irradiated with light emitted by said first light source via said first optical fiber,
said light incident point on the surface of said subject is irradiated with light emitted by said second light source via said second optical fiber in a direction different from that of the light of said first wavelength, reflected light of the light of said first wavelength reflected at said light incident point and scattered light of the light of said second wavelength are incident on said first photodetector without via fiber,
reflected light of the light of said second wavelength reflected at said light incident point and scattered light of the light of said first wavelength are incident on said second photodetector without via fiber,
said third optical fiber has an incident end thereof disposed at such a position as to be in contact with the surface of said subject in an area spaced apart from said light incident point on the subject surface, and
said third photodetector is adapted to receive light exiting from an area spaced apart from said light incident point on the surface of the subject via said third optical fiber.

12. The blood sugar level measuring apparatus according to claim 11, wherein a plane of incidence of the light of said first wavelength and a plane of incidence of the light of said second wavelength are substantially perpendicular to each other with respect to said light incident point on the surface of said subject.

13. The blood sugar level measuring apparatus according to claim 11, wherein an exiting end of said first optical fiber, an exiting end of said second optical fiber, a receiving plane of said first photodetector, and a receiving plane of said second photodetector are disposed in the vicinity of a circular conical surface with an apex located at said light incident point.

14. The blood sugar level measuring apparatus according to claim 11, wherein an incident end of said third optical fiber is located on a plane of incidence of the light of said first wavelength, or a plane of incidence of the light of said second wavelength.

15. The blood sugar level measuring apparatus according to claim 11, wherein an incident end of said third optical fiber is located on a plane that forms an angle of approximately 45° with a plane of incidence of the light of said first wavelength and with a plane of incidence of the light of said second wavelength.

16. The blood sugar level measuring apparatus according to claim 11, wherein said first wavelength is a wavelength at which the molar absorption coefficient of oxyhemoglobin and that of reduced hemoglobin are equal, and said second wavelength is a wavelength for detecting a difference in absorbance between oxyhemoglobin and reduced hemoglobin.

17. The blood sugar level measuring apparatus according to claim 11, wherein
said optical measurement portion further comprises a control portion for controlling the emission of light from said first light source and said second light source, said control portion causing said first light source and said second light source to emit light alternately such that said light incident point on the surface of the subject is irradiated with the light of said first wavelength and the light of said second wavelength alternately,
reflected light of said first wavelength from said light incident point is mainly incident on said first photodetector when said first light source is emitting, and scattered light of the light of said second wavelength is mainly incident when said second light source is emitting,
reflected light of said second wavelength from said light incident point on the surface of the subject is mainly incident on said second photodetector when said second light source is emitting, and scattered light of the light of said first wavelength is mainly incident when said first light source is emitting.

18. The blood sugar level measuring apparatus according to claim 11, wherein the reflected light of the light of said first wavelength reflected at said light incident point and the scattered light of the light of said second wavelength are directly incident on said first photodetector, and the reflected light of the light of said second wavelength reflected at said light incident point and the scattered light of the light of said first wavelength are directly incident on said second photodetector.

19. A blood sugar level measuring apparatus comprising:
an ambient temperature detector for measuring ambient temperature;
a body-surface contact portion with which a body surface comes into contact;
a radiation temperature detector for measuring radiation heat from said body surface;
an adjacent temperature detector disposed adjacent to said body-surface contact portion;
an indirect temperature detector for detecting the temperature at a position spaced apart from said body-surface contact portion;
a heat conducting member connecting said body-surface contact portion and said indirect temperature detector;
a light source for producing light of at least two different wavelengths;
an optical system for irradiating the body surface with light emitted by said light source;
at least three different photodetectors for detecting the light that has been irradiated onto the body surface;
a memory portion in which relationships between individual outputs from said ambient temperature detector, said radiation temperature detector, said adjacent temperature detector, said indirect temperature detector, and said at least three different photodetectors, and blood sugar levels are stored;
a calculation portion for calculating a blood sugar level by applying said individual outputs to said relationships stored in said memory portion; and
a display portion for displaying the result of calculation in said calculation portion, wherein
said light source comprises a first light source for producing light of a first wavelength and a second light source for producing light of a second wavelength,
said optical system comprises a first optical fiber and a second optical fiber,
said photodetectors comprises a first photodetector, a second photodetector, and a third photodetector that receives light via a third optical fiber, wherein
light emitted by said first light source is irradiated onto a light incident point on the surface of a subject via said first optical fiber,
light emitted by said second light source is irradiated onto said light incident point on the subject surface via said second optical fiber in a direction different from the light of said first wavelength,
reflected light of the light of said first wavelength reflected at said light incident point and scattered light of the light of said second light are incident on said first photodetector without via fiber,
reflected light of the light of said second wavelength reflected at said light incident point and scattered light of the light of said first wavelength are incident on said second photodetector without via fiber,
said third optical fiber has an incident end thereof located at such a position as to be in contact with the subject surface in an area spaced apart from said light incident point on the subject surface, and said third photodetector is adapted to receive, via said third optical fiber, light exiting from an area spaced apart from said light incident point on the subject surface.

20. The blood sugar level measuring apparatus according to claim 19, wherein the reflected light of the light of said first wavelength reflected at said light incident point and the scattered light of the light of said second wavelength are directly incident on said first photodetector, and the reflected light of the light of said second wavelength reflected at said light incident point and the scattered light of the light of said first wavelength are directly incident on said second photodetector.
